(19) United States
(12) Patent Application Publication
(10) Pub. No.: US 2011/0159600 A1
(43) Pub. Date: Jun. 30, 2011

(54) NATRIURETIC PEPTIDES AND ADIPONECTIN IN SUBJECTS WITH A METABOLIC SYNDROME

(76) Inventors: Georg Hess, Mainz (DE); Andrea Horsch, Mannheim (DE); Dietmar Zdunek, Tutzing (DE)

(21) Appl. No.: 13/041,519

(22) Filed: Mar. 7, 2011

Related U.S. Application Data
(63) Continuation of application No. PCT/EP2009/061723, filed on Sep. 10, 2009.

(30) Foreign Application Priority Data
Sep. 11, 2008 (EP) 08164125.0

(51) Int. Cl. G01N 33/50; B01J 19/00

(52) U.S. Cl. 436/86; 422/500

(57) ABSTRACT

The present invention is concerned with a method for predicting the risk of mortality and/or a cardiovascular event in a subject who suffers from the metabolic syndrome based on the determination of a natriuretic peptide and adiponectin in a sample of a subject. Moreover, the present invention relates to a method for identifying a subject being susceptible to a therapy that intends to increase the level of adiponectin in a subject based on the determination of the aforementioned markers. Further disclosed are kits and devices adapted to carry out the method of the present invention.

### RELATED APPLICATIONS

This application is a continuation of PCT/EP2009/061723 filed Sep. 10, 2009 and claims priority to EP 08164125.0 filed Sep. 11, 2008.

### FIELD OF THE INVENTION

The present invention is concerned with a method for predicting the risk of mortality and/or a cardiovascular event in a subject who suffers from the metabolic syndrome based on the determination of a natriuretic peptide and adiponectin in a sample of a subject. Moreover, the present invention relates to a method for identifying a subject being susceptible to a therapy that intends to increase the level of adiponectin in a subject based on the determination of the aforementioned markers. Further comprised by the present invention are kits and devices adapted to carry out the method of the present invention.

### BACKGROUND OF THE INVENTION

The metabolic syndrome is associated with an imbalance between energy intake and the capacity for energy storage and results in the ectopic deposition of lipids in visceral fat, liver, skeletal muscle, pancreatic beta cells and vessel walls (Smith (2006) Obesity Vol. 14 Suppl. 128S-134S). It is a constellation of interrelated risk factors of metabolic origin that is considered to directly promote the development of atherosclerotic cardiovascular disease and diabetes type 2 (Grundy et al. (2005) Circulation 112, 2735-2752).

The predominant underlying risk factors for the syndrome are, presumably, abdominal obesity, insulin resistance. Other conditions that are associated with the metabolic syndrome can be physical inactivity and hormonal imbalances. The metabolic syndrome can occur both in obese and in non-obese patients. In non-obese patients, lipids are stored in visceral reservoirs. Once these reservoirs are filled, lipids are also stored in other tissues and organs. Especially, in the presence of visceral obesity, there is a significantly increased risk of progressing to diabetes type 2 and cardiovascular disease. It is generally believed that the metabolic syndrome is driving the twin global epidemics of diabetes and cardiovascular disease. The prevalence of metabolic syndrome is assumed to be approximately between 20 to 25% of the population in industrialized countries.

A major hormone that is involved in the development of the metabolic syndrome is adiponectin. Adiponectin is the most abundant adipokine secreted by adipocytes. Adipocytes are endocrine secretory cells which release free fatty acids and produce, in addition to adiponectin, several cytokines such as tumor necrosis factor (TNF) alpha, leptin, and interleukins

It is generally assumed that adiponectin sensitizes the body to insulin. Decreased adiponectin levels are observed in patients with diabetes and metabolic syndrome and are thought to play a key role in insulin resistance (see e.g. Han et al. Journal of the American College of Cardiology, Vol. 49(5)531-8). infarction among men without previous cardiovascular disease. A In humans, extensive studies of the metabolic actions of adiponectin, particularly with respect to cardiovascular disease and type 2 diabetes mellitus have been carried within the last decade. The data in the scientific literature regarding the relationship between the adiponectin level and the outcome are, however, very inconsistent. While some groups report that subjects with low adiponectin levels are at reduced risk of suffering from cardiovascular events, other groups report that subjects with increased amounts of adiponectin are at increased risk of cardiovascular events. For example, Pischon et al. (JAMA, 2004; 291:1730-1737) found that, over a follow-up period of 6 years, high plasma adiponectin levels are associated with lower risk of myocardial study of Kumada et al. (Arterioscler. Thromb. Vasc. Biol. 2003; 23:85-89) suggested that male patients with hypoadiponectinemia (lower than 4.0 g/ml) had a 2-fold increase in CAD prevalence, independent of well-known CAD risk factors. In agreement with Kumada and Pischon, Inoue et. al. (Am J Cardiol. 2007 Aug. 15; 100(4):569-74) showed that low HMW adiponectin levels predicted cardiovascular events during a follow-up of seven years. Contrarily, however, Pilz et al. (Journal of Clinical Endocrinology & Metabolism 91 (11):4277-4286) showed that high adiponectin independently predicts all-cause cardiovascular and non-cardiovascular mortality in subjects with coronary artery disease (CAD). Also, Tamura et al. (Circ. J. 2007; 71: 623-630) showed that high adiponectin levels are an independent predictor of mortality in patients with congestive heart failure. Studies of Kistorp et al. (Circulation, 2005;112, 1756-1762) suggested that increased adiponectin and NT-proBNP levels are associated with increased mortality in heart failure patients. Furthermore, a study of Haugen et al. (International Journal of Cardiology, 2008, 125: 216-219) indicated that adiponectin and NT-proBNP levels were increased in patients >70 years with severe heart failure. Also, Tsutamoto et al. (European Heart Journal, 2007, 28: 1723-1730) showed that high molecular weight adiponectin, total adiponectin and NT-proBNP are independent predictors in patients with chronic heart failure. Lawlor et al., however, showed that adiponectin does not predict coronary heart disease in woman at all (J Clin Endocrinol Metab 2005; 90: 5677-5683).

Adiponectin levels decrease with obesity and are significantly lower in patients with the metabolic syndrome than in healthy individuals (Salmenniemi U et al. Multiple abnormalities in glucose and energy metabolism and coordinated changes in levels of adiponectin, cytokines, and adhesion molecules in subjects with metabolic syndrome Circulation 2004; 110:3842-3848). In many studies, adiponectin has been consistently correlated with several components of the metabolic syndrome. It was shown, that the adiponectin level decreases when abdominal adiposity, plasma glucose, haemoglobin A1c and insulin resistance increase (Lara-Castro et al, Current Opinion in Lipidology, 2007, 18:263-270).

Increasing adiponectin levels have been suggested as promising therapeutic strategy for the treatment of insulin resistance, metabolic syndrome and type 2 diabetes (see, e.g. Kadowaki et al.(2006) Adiponectin and adiponectin receptors in insulin resistance, diabetes, and the metabolic syndrome. J Clin Invest. 116(7): 1784-1792). Recently, it was shown that thiazolidinediones significantly increase the adiponectin level. This effect may be an important component of the drug action of thiazolidinediones (see Lara-Castro et al., loc. cit.). However, it not known yet whether patients generally will benefit from a therapy that aims to increase the adiponectin level.

Natriuretic peptides, particularly brain natriuretic peptides, are well established markers in heart disease, particularly in chronic heart failure. Generally, the higher the concentration of BNP and/or NT-proBNP is, the worse is the outcome. WO 02/083913 discloses that the brain natriuretic peptide (BNP) and variants thereof may be used to predict near-term mortality and morbidity in patients suffering already from heart failure. In an attempt to diagnose cardiovascular diseases, in particular myocardial ischemia, WO 02/089657 also discloses various biomarkers which may be used as prognostic indicators for the diseases and disorders once diagnosed. Among others, BNP is mentioned as a suitable biomarker. Moreover, Hutfless et al. have shown that based on the BNP concentration in patient blood after cardiac surgery, it shall be possible to predict morbidity or mortality within a 30 day period after the surgery (Hutfless 2004, Utility of B-type natriuretic peptide in predicting postoperative complications and outcomes in patients undergoing heart surgery. J Am Coll Cardiol 43: 1873-9). J Am Coll Cardiol 45: 1043-50).

Early identification of subjects suffering from the metabolic syndrome which are at increased risk of mortality or cardiovascular events is highly desirable. However, reliable method for identifying those subjects at risk have not been described yet.

The technical problem underlying the present invention can be seen as the provision of means and methods for complying with the aforementioned needs. The technical problem is solved by the embodiments characterized in the claims and herein below.

### SUMMARY OF THE INVENTION

Accordingly, the present invention relates to a method for predicting the risk of mortality and/or a cardiovascular event in a subject suffering from the metabolic syndrome comprising the steps of:
a) determining, in a sample of the subject, the amount of adiponectin,
b) determining, in a sample of the subject, the amount of natriuretic peptide,
c) comparing the amounts as determined in step a) and b) to reference amounts, and
d) predicting the risk of mortality and/or a cardiovascular event in the subject based on the information obtained in step c).

The method of the present invention, preferably, is an in vitro method. Moreover, it may comprise steps in addition to those explicitly mentioned above. For example, further steps may relate to sample pre-treatments or evaluation of the results obtained by the method. The method of the present invention may be also used for monitoring, confirmation, and subclassification. The method may be carried out manually and/or assisted by automation. Preferably, step (a), (b), (c) and/or (d) may in total or in part be assisted by automation, e.g., by a suitable robotic and sensory equipment for the determination in step (a) and/or (b) or a computer-implemented comparison in step (c).

### DETAILED DESCRIPTION OF THE INVENTION

The term “subject” as used herein relates to animals, preferably mammals, and, more preferably, humans. However, it is envisaged by the present invention that the subject shall suffer from the metabolic syndrome.

The term “metabolic syndrome” is well known by the skilled person. As used herein the term, preferably, relates to a cluster of risk factors including hypertriglyceridemia, abdominal obesity, arterial hypertony, and various metabolic disorders including dyslipidaemia and hyperglycemia. In the art, different terms are known for the metabolic syndrome such as metabolic syndrome X, syndrome X, insulin resistance syndrome, and Reaven's syndrome. It is also known that various criteria exist for identifying individuals having the metabolic syndrome. Preferably, the metabolic syndrome as used herein is defined by the criteria according to the WHO (World Health Organization, see also Definition, Diagnosis and Classification of Diabetes Mellitus and its Complications; Part 1: Diagnosis and Classification of Diabetes Mellitus. Geneva: WHO Department of Noncommunicable Disease Surveillance; 1999), or by the criteria according to the EGIR (European Group for the Study of Insulin Resistance), or by the criteria according to the NCEP (National Education Program Adult Treatment Panel III, frequently also referred to as ATP III criteria) or by the AHA/NHLBI criteria (American Heart Association/Updated NCEP). Of these the EGIR criteria are more preferred, and the ATP III criteria are most preferred for the diagnosis of a metabolic syndrome. The various criteria defined by the organizations are known in the art (see, for a review Scott M. Grundy et al., Circulation; 2005(112):2735-2752 which hereby is incorporated by reference in its entirety with respect to the disclosure content). In the context of the present invention, a subject who fulfils the specific criteria as defined by the various organizations as referred to herein suffers from the metabolic syndrome, and, thus, has developed a metabolic syndrome.

The criteria according to the WHO (1999) require, for the diagnosis of a metabolic syndrome in a subject, the presence of insulin resistance identified by one of the following: diabetes mellitus, impaired glucose tolerance, impaired fasting glucose or for those with normal fasting glucose levels (<110 mg/dL) glucose uptake below the lowest quartile for background population under investigation under hyperinsulinemic, euglycemic conditions. In addition to insulin resistance, the presence at least two of the following is required:
a) blood pressure: ≧140 mmHg systolic or ≧90 mmHg diastolic (or on treatment for high blood pressure)
b) plasma triglycerides (TG): ≧1.695 mmol/L and high-density lipoprotein cholesterol (HDL C) ≦0.9 mmol/L (for male subjects), ≦1.0 mmol/L (for female subjects)
c) waist to hip ratio >0.90 (for male subjects); >0.85 (for female subjects), and/or body mass index (BMI) >30 kg/m²
d) urinary albumin excretion ratio ≧20 μg/min or albumin to creatinine ratio ≧30 mg/g.

The US National Cholesterol Education Program Adult Treatment Panel III [ATP-III/NCEP, 2001, see also: Expert Panel on Detection, Evaluation, and Treatment of High Blood Cholesterol in Adults. Executive Summary of The Third Report of The National Cholesterol Education Program (NCEP) Expert Panel on Detection, Evaluation, And Treatment of High Blood Cholesterol In Adults (Adult Treatment Panel III). JAMA 2001;285:2486-97, or National Cholesterol Education Program (NCEP) Expert Panel on Detection, Evaluation, and Treatment of High Blood Cholesterol in Adults (Adult Treatment Panel III) Third report of the National Cholesterol Education Program (NCEP) Expert Panel on Detection, Evaluation, and Treatment of High Blood Cholesterol in Adults (Adult Treatment Panel III) final report Circulation 2002; 106:3143-3421] requires, for the presence/diagnosis of a metabolic syndrome, at least three of the following:
a) abdominal obesity given as waist circumference ≧102 cm (for male subjects), ≧88 cm (for female subjects)
b) plasma triglycerides ≧1.695 mmol/L (150 mg/dl)
c) HDL-C ≦40 mg/dL (for male subjects), ≦50 mg/dL (for female subjects)
d) blood pressure ≧130/≧85 mmHg
e) fasting plasma glucose ≧6.1 mmol/L (110 mg/dl).

The European Group for the Study of Insulin Resistance (EGIR) requires for the diagnosis/presence of a metabolic syndrome: insulin resistance defined as the top 25% of the fasting insulin values among non-diabetic individuals, and two or more of the following [see also Balkau B, Charles M A, European Group for the Study of Insulin Resistance (EGIR) Comment on the provisional report from the WHO consultation, Diabet Med 1999; 16:442-443]:
a) central obesity: waist circumference ≧94 cm (male), ≧80 cm (female) and/or body mass index (BMI) ≦30 kg/m2
b) plasma triglycerides ≧150 mg/dL and/or HDL-C <39 mg/dL (or treated for dyslipidaemia)
c) hypertension: blood pressure ≧140/90 mm Hg (or antihypertensive medication)
d) fasting plasma glucose ≧6.1 mmol/L

The AHA/NHLBI criteria (American Heart Association/Updated NCEP, see also Grundy S M, Brewer H B, Cleeman J I, Smith S C, Lenfant D, for the Conference Participants. Definition of metabolic syndrome: report of the National, Heart, Lung, and Blood Institute/American Heart Association conference on scientific issues related to definition. Circulation. 2004; 109:433-438) require, for the presence/diagnosis of a metabolic syndrome, at least three of the following: elevated waist circumference: (Men: Equal to or greater than 102 cm, women equal to or greater than 88 cm); elevated triglycerides (equal to or greater than 150 mg/dl); reduced HDL cholesterol (men: less than 40 mg/dL; women: less than 50 mg/dL); elevated blood pressure (equal to or greater than 130/85 mm Hg or use of medication for hypertension) elevated fasting glucose (equal to or greater than 100 mg/dL (5.6 mmol/L) or use of medication for hyperglycemia).

The term “predicting” used herein refers to assessing the probability according to which a subject suffering from the metabolic syndrome will die (e.g. mortality caused by the heart failure) and/or develop a cardiovascular event, preferably an acute cardiovascular event such as an acute coronary syndrome (ACS) within a defined time window (predictive window) in the future. The predictive window is an interval in which the subject will develop a cardiovascular event or will die according to the predicted probability. The predictive window may be the entire remaining lifespan of the subject upon analysis by the method of the present invention. Preferably, however, the predictive window is an interval of one month, six months or one, two, three, four, five, eight or ten years after the method of the present invention has been carried out (more preferably and precisely, after the sample to be analyzed by the method of the present invention has been obtained). Most preferably, the predictive window is an interval of eight years. As will be understood by those skilled in the art, such an assessment is usually not intended to be correct for 100% of the subjects to be analyzed. The term, however, requires that the assessment will be valid for a statistically significant portion of the subjects to be analyzed. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the present invention allows that the prediction will be correct for at least 60%, at least 70%, at least 80%, or at least 90% of the subjects of a given cohort.

The term “mortality” as used herein relates to mortality from any cause, preferably, from a cardiovascular complication or cardiovascular event. The term “cardiovascular event” as used herein refers to any disorder of the cardiovascular system including preferably any acute cardiovascular event. Acute cardiovascular events are, preferably, stable angina pectoris (SAP) or acute coronary syndrome (ACS). ACS patients can show unstable angina pectoris (UAP) or myocardial infarction (MI). MI can be an ST-elevation MI (STEMI) or a non-ST-elevation MI (NSTEMI). NSTE-ACS as used herein encompasses UAP and NSTEMI. The occurring of an MI can be followed by a left ventricular dysfunction (LVD), development of heart failure or even mortality. Further preferred cardiovascular events encompass cardiac brady- or tachyarrhythmias including sudden cardiac death and stroke (cerebrovascular events or accidents). Also, mortality can also refer to the death rate or the ratio of number of deaths to a given population of subjects.

The expression “predicting the risk of mortality and/or of a cardiovascular event” as used herein means that if the subject to be analyzed by the method of the present invention is allocated either into the group of subjects of a population having a normal, i.e. average, risk for developing an acute cardiovascular event or mortality, or into a group of subjects having a elevated risk, or into a group having a reduced risk. An elevated risk as referred to in accordance with the present invention, preferably, means that the risk of developing a cardiovascular event or the risk of mortality within a predetermined predictive window is elevated significantly (i.e. increased significantly) for a subject with respect to the average risk for a cardiovascular event or cardiac mortality in a population of subjects (with the metabolic syndrome). A reduced risk as referred to in accordance with the present invention, preferably, means that the risk of developing a cardiovascular event or the risk of mortality within a predetermined predictive window is reduced significantly for a subject with respect to the average risk for a cardiovascular event or cardiac mortality in a population of subjects with the metabolic syndrome. Particularly, a significant increase or reduction of a risk is an increase or reduction or a risk of a size which is considered to be significant for prognosis, particularly the increase or reduction is considered statistically significant. The terms “significant” and “statistically significant” are known by the person skilled in the art. Thus, whether an increase or reduction of a risk is significant or statistically significant can be determined without further ado by the person skilled in the art using various well known statistic evaluation tools.

Preferably, for a predictive window of eight years, the average risk of mortality is within the range of 10.0% and 19.0%, more preferably within the range of 12.0% to 17.0%, most preferably, within the range of 14.0% to 16.0%. A reduced risk of mortality as used herein, preferably, relates to a risk of less than 10%, and, more preferably, a risk within the range of 5.0% and 10.0%, preferably with respect to a predictive window of eight years. An elevated, and, thus increased risk of mortality as used herein, preferably, relates to a risk of more than 19%, even more preferably, more than 20%, and, most preferably, a risk within the range of 20.0% and 30.0%, with respect to a predictive window of eight years.

The term “sample” refers to a sample of a body fluid, to a sample of separated cells or to a sample from a tissue or an organ. Samples of body fluids can be obtained by well known techniques and include, preferably, samples of blood, plasma, serum, or urine, more preferably, samples of blood, plasma or serum. Tissue or organ samples may be obtained from any tissue or organ by, e.g., biopsy. Separated cells may be obtained from the body fluids or the tissues or organs by separating techniques such as centrifugation or cell sorting. Preferably, cell-, tissue- or organ samples are obtained from those cells, tissues or organs which express or produce the peptides referred to herein.

The term “natriuretic peptide” comprises Atrial Natriuretic Peptide (ANP)-type and Brain Natriuretic Peptide (BNP)-type peptides and variants thereof having the same predictive potential. Natriuretic peptides according to the present invention comprise ANP-type and BNP-type peptides and variants thereof (see e.g. Bonow, 1996, Circulation 93: 1946-1950). ANP-type peptides comprise pre-proANP, proANP, NT-proANP, and ANP. BNP-type peptides comprise pre-proBNP, proBNP, NT-proBNP, and BNP. The pre-pro peptide (134 amino acids in the case of pre-proBNP) comprises a short signal peptide, which is enzymatically cleaved off to release the pro peptide (108 amino acids in the case of proBNP). The pro peptide is further cleaved into an N-terminal pro peptide (NT-pro peptide, 76 amino acids in case of NT-proBNP) and the active hormone (32 amino acids in the case of BNP, 28 amino acids in the case of ANP). Preferably, natriuretic peptides according to the present invention are NT-proANP, ANP, and, more preferably, NT-proBNP, BNP, and variants thereof. ANP and BNP are the active hormones and have a shorter half-life than their respective inactive counterparts, NT-proANP and NT-proBNP. BNP is metabolised in the blood, whereas NT-proBNP circulates in the blood as an intact molecule and as such is eliminated renally. The in-vivo half-life of NTproBNP is 120 min longer than that of BNP, which is 20 min (Smith 2000, J Endocrinol. 167: 239-46.). Preanalytics are more robust with NT-proBNP allowing easy transportation of the sample to a central laboratory (Mueller 2004, Clin Chem Lab Med 42: 942-4.). Blood samples can be stored at room temperature for several days or may be mailed or shipped without recovery loss. In contrast, storage of BNP for 48 hours at room temperature or at 4° Celsius leads to a concentration loss of at least 20% (Mueller loc.cit.; Wu 2004, Clin Chem 50: 867-73.). Therefore, depending on the time-course or properties of interest, either measurement of the active or the inactive forms of the natriuretic peptide can be advantageous. The most preferred natriuretic peptides according to the present invention are NT-proBNP or variants thereof. As briefly discussed above, the human NT-proBNP, as referred to in accordance with the present invention, is a polypeptide comprising, preferably, 76 amino acids in length corresponding to the N-terminal portion of the human NT-proBNP molecule. The structure of the human BNP and NT-proBNP has been described already in detail in the prior art, e.g., WO 02/089657, WO 02/083913 or Bonow loc. cit. Preferably, human NT-proBNP as used herein is human NT-proBNP as disclosed in EP 0 648 228 B1. These prior art documents are herewith incorporated by reference with respect to the specific sequences of NT-proBNP and variants thereof disclosed therein. The NT-proBNP referred to in accordance with the present invention further encompasses allelic and other variants of the specific sequence for human NT-proBNP discussed above.

Specifically, envisaged are variant polypeptides which are on the amino acid level preferably, at least 50%, 60%, 70%, 80%, 85%, 90%, 92%, 95%, 97%, 98%, or 99% identical to human NTproBNP (preferably over the entire length of human NT-proBNP). The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Variants referred to above may be allelic variants or any other species specific homologs, paralogs, or orthologs. Substantially similar and also envisaged are proteolytic degradation products which are still recognized by the diagnostic means or by ligands directed against the respective full-length peptide. Also encompassed are variant polypeptides having amino acid deletions, substitutions, and/or additions compared to the amino acid sequence of human NT-proBNP as long as the polypeptides have NT-proBNP properties. NT-proBNP properties as referred to herein are immunological and/or biological properties. Also included by biological properties are the properties of the NT-proBNP or preproBNP precursor polypeptide and the properties of its other subfragments. Accordingly, a variant NT-proBNP includes a N-terminal fragment of a proBNP or preproBNP molecule having essentially the same biological properties than the preproBNP molecule which gives rise to the above specified NT-proBNP. The biological properties of the subfragments include the biological activities of the mature BNP molecule including its vasorelaxant activity. Thus, a variant NT-proBNP can be derived from a variant proBNP or preproBNP which on the one hand gives rise to the variant NT-proBNP and on the other hand to the variant mature BNP having at least vasorelaxant activity.

Whether a variant has vasorelaxant activity or not can be determined by methods well known in the art (see e.g. El Bardei et al. Planta Med 2003; 69: 75-77 or Morel et al., J Cardiovasc. Pharmacol 1994; 24: 524 ff). Preferably, vasorelaxant activity can be assessed by measuring aortic contraction in the presence and the absence of the variant. Aortic contraction and, thus, vasorelaxant activity is preferably assessed as follows: aortic segments (preferably from rats or rabbits) are suspended in organ baths filled with a physiological solution (NaCl: 122 mM; KCl 5.9 mM; NaHCO3: 15 mM; MgCl2 1.25 mM; CaCl2, 1.25 mM; glucose 11 mM) bubbled with a gas mixture of 95% 02, 5% CO2 and maintained at 37° C. Contraction is evoked by changing the physiological solution to a depolarizing 100 mM KCl solution (NaCl 27 mM; KCl 100mM; NaHCO3 15 mM, MgCl2 1.25 mM; CaCl2, 1.25 mM; glucose 11 mM). The amplitude of the contraction evoked in the presence of the tested variant is then compared to the response measured in its absence. Verapamil can be used as a reference compound.

Preferably, the NT-proBNP variants have immunological properties (i.e. epitope composition) comparable to those of NT-proBNP. Thus, the variants shall be recognizable by the aforementioned means or ligands used for determination of the amount of the natriuretic peptides. The biological and/or immunological NT-proBNP properties can be detected, preferably, by the assay described in Karl et al. (Karl 1999, Scand J Clin Invest 230:177-181) or Yeo et al. (Yeo 2003, Clinica Chimica Acta 338:107-115). Variants also include posttranslationally modified peptides such as glycosylated peptides. Further, a variant in accordance with the present invention is also a peptide or polypeptide which has been modified after collection of the sample, for example by covalent or non-covalent attachment of a label, particularly a radioactive or fluorescent label, to the peptide.

Adiponectin is a polypeptide (one of several known adipocytokines) secreted by the adipocyte. In the art, adiponectin is frequently also referred to as Acrp30 and apM1. Adiponectin has recently been shown to have various activities such as anti-inflammatory, antiatherogenic, preventive for metabolic syndrome, and insulin sensitizing activities. Adiponectin is encoded by a single gene, and has 244 amino acids, the molecular weight is approximately 30 kilodaltons. The mature human adiponectin protein encompasses amino acids 19 to 244 of full-length adiponectin. A globular domain is thought to encompass amino acids 107-244 of full-length adiponectin. The sequence of the adiponectin polypeptide is well known in the art, and, e.g., disclosed in WO2008/084003.

Adiponectin associates itself into larger structures. Three adiponectin polypeptides bind together and form a homotrimer. These trimers bind together to form hexamers or dodecamers. Adiponectin is known to exist in a wide range of multimer complexes in plasma and combines via its collagen domain to create 3 major oligomeric forms: a low-molecular weight (LMW) trimer, a middle-molecular weight (MMW) hexamer, and high-molecular weight (HMW) 12- to 18-mer adiponectin (Kadowaki et al. (2006) Adiponectin and adiponectin receptors in insulin resistance, diabetes, and the metabolic syndrome. J Clin Invest. 116(7): 1784-1792; Rexford S. Ahima, Obesity 2006; 14:2425-2495). Adiponectin has been reported to have several physiological actions, such as protective activities against atherosclerosis, improvement of insulin sensitivity, and prevention of hepatic fibrosis.

Adiponectin as used herein, preferably, relates to low molecular weight adiponectin, mid molecular weight adiponectin, more preferably, to total adiponectin, and, most preferably, to high molecular weight adiponectin. The terms high molecular weight adiponectin (12- to 18-mer adiponectin, preferably, 18-mer adiponectin), low and mid molecular weight adiponectin and total adiponectin are understood by the skilled person. Preferably, the adiponectin is human adiponectin. Methods for the determination of adiponectin are, e.g., disclosed in US 2007/0042424 A1 as well as in WO 2008/084003. Preferably, the amount of adiponectin is determined in a serum sample.

The adiponectin referred to in accordance with the present invention further encompasses allelic and other variants of the specific sequence for human adiponectin discussed above. Specifically, envisaged are variant polypeptides which are on the amino acid level preferably, at least 50%, 60%, 70%, 80%, 85%, 90%, 92%, 95%, 97%, 98%, or 99% identical to human adiponectin (preferably over the entire length of human adiponectin). The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Variants referred to above may be allelic variants or any other species specific homologs, paralogs, or orthologs. Substantially similar and also envisaged are proteolytic degradation products which are still recognized by the diagnostic means or by ligands directed against the respective full-length peptide. Also encompassed are variant polypeptides having amino acid deletions, substitutions, and/or additions compared to the amino acid sequence of human adiponectin. Preferably, the adiponectin variants have adiponectin properties, preferably, including its ability to reduce adipose tissue which can be tested in known animal models.

Whether a variant of adiponectin is capable of reducing adipose tissue can be, e.g., tested as follows: The tested variant can be administered to adipocytes, to tissue comprising adipocytes or to an non-human animal. Preferably, a variant of adiponectin is capable of reducing adipose tissue, if the amount of fat comprised by the adipocytes, the tissue or the non-human animal is decreased as compared with control adipocytes, a control tissue or not-human animal which have not been contacted with the variant. Preferably, the non-human animal will be sacrificed after the determining whether the variant reduces adipose tissues (It is, thus, to be understood that such a determination is not deemed to be a method of treatment of the human or animal body).

Determining the amount of adiponectin or of a natriuretic peptide or any other peptide or polypeptide referred to in this specification relates to measuring the amount or concentration, preferably semi-quantitatively or quantitatively. Measuring can be done directly or indirectly. Direct measuring relates to measuring the amount or concentration of the peptide or polypeptide based on a signal which is obtained from the peptide or polypeptide itself and the intensity of which directly correlates with the number of molecules of the peptide present in the sample. Such a signal—sometimes referred to herein as intensity signal—may be obtained, e.g., by measuring an intensity value of a specific physical or chemical property of the peptide or polypeptide. Indirect measuring includes measuring of a signal obtained from a secondary component (i.e. a component not being the peptide or polypeptide itself) or a biological read out system, e.g., measurable cellular responses, ligands, labels, or enzymatic reaction products.

In accordance with the present invention, determining the amount of a peptide or polypeptide can be achieved by all known means for determining the amount of a peptide in a sample. The means comprise immunoassay devices and methods which may utilize labeled molecules in various sandwich, competition, or other assay formats. The assays will develop a signal which is indicative for the presence or absence of the peptide or polypeptide. Moreover, the signal strength can, preferably, be correlated directly or indirectly (e.g. reverse-proportional) to the amount of polypeptide present in a sample. Further suitable methods comprise measuring a physical or chemical property specific for the peptide or polypeptide such as its precise molecular mass or NMR spectrum. The methods comprise, preferably, biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays (available for example on ELECSYS analyzers), CBA (an enzymatic Cobalt Binding Assay, available for example on Roche-Hitachi analyzers), and latex agglutination assays (available for example on Roche-Hitachi analyzers).

Preferably, determining the amount of a peptide or polypeptide comprises the steps of (a) contacting a cell capable of eliciting a cellular response the intensity of which is indicative of the amount of the peptide or polypeptide with the peptide or polypeptide for an adequate period of time, (b) measuring the cellular response. For measuring cellular responses, the sample or processed sample is, preferably, added to a cell culture and an internal or external cellular response is measured. The cellular response may include the measurable expression of a reporter gene or the secretion of a substance, e.g. a peptide, polypeptide, or a small molecule. The expression or substance shall generate an intensity signal which correlates to the amount of the peptide or polypeptide.

Also preferably, determining the amount of a peptide or polypeptide comprises the step of measuring a specific intensity signal obtainable from the peptide or polypeptide in the sample. As described above, such a signal may be the signal intensity observed at an m/z variable specific for the peptide or polypeptide observed in mass spectra or a NMR spectrum specific for the peptide or polypeptide.

Determining the amount of a peptide or polypeptide may, preferably, comprises the steps of (a) contacting the peptide with a specific ligand, (b) (optionally) removing non-bound ligand, (c) measuring the amount of bound ligand. The bound ligand will generate an intensity signal. Binding according to the present invention includes both covalent and non-covalent binding. A ligand according to the present invention can be any compound, e.g., a peptide, polypeptide, nucleic acid, or small molecule, binding to the peptide or polypeptide described herein. Preferred ligands include antibodies, nucleic acids, peptides or polypeptides such as receptors or binding partners for the peptide or polypeptide and fragments thereof comprising the binding domains for the peptides, and aptamers, e.g. nucleic acid or peptide aptamers. Methods to prepare such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g. phage display. Antibodies as referred to herein include both polyclonal and monoclonal antibodies, as well as fragments thereof, such as Fv, Fab and F(ab)2 fragments that are capable of binding antigen or hapten. The present invention also includes single chain antibodies and humanized hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art. Preferably, the ligand or agent binds specifically to the peptide or polypeptide. Specific binding according to the present invention means that the ligand or agent should not bind substantially to (“cross-react” with) another peptide, polypeptide or substance present in the sample to be analyzed. Preferably, the specifically bound peptide or polypeptide should be bound with at least 3 times higher, more preferably at least 10 times higher and even more preferably at least 50 times higher affinity than any other relevant peptide or polypeptide. Non-specific binding may be tolerable, if it can still be distinguished and measured unequivocally, e.g. according to its size on a Western Blot, or by its relatively higher abundance in the sample. Binding of the ligand can be measured by any method known in the art. Preferably, the method is semi-quantitative or quantitative. Suitable methods are described in the following.

First, binding of a ligand may be measured directly, e.g. by NMR or surface plasmon resonance. Second, if the ligand also serves as a substrate of an enzymatic activity of the peptide or polypeptide of interest, an enzymatic reaction product may be measured (e.g. the amount of a protease can be measured by measuring the amount of cleaved substrate, e.g. on a Western Blot). Alternatively, the ligand may exhibit enzymatic properties itself and the “ligand/peptide or polypeptide” complex or the ligand which was bound by the peptide or polypeptide, respectively, may be contacted with a suitable substrate allowing detection by the generation of an intensity signal. For measurement of enzymatic reaction products, preferably the amount of substrate is saturating. The substrate may also be labeled with a detectable lable prior to the reaction. Preferably, the sample is contacted with the substrate for an adequate period of time. An adequate period of time refers to the time necessary for an detectable, preferably measurable, amount of product to be produced. Instead of measuring the amount of product, the time necessary for appearance of a given (e.g. detectable) amount of product can be measured.

Third, the ligand may be coupled covalently or non-covalently to a label allowing detection and measurement of the ligand. Labeling may be done by direct or indirect methods. Direct labeling involves coupling of the label directly (covalently or non-covalently) to the ligand. Indirect labeling involves binding (covalently or non-covalently) of a secondary ligand to the first ligand. The secondary ligand should specifically bind to the first ligand. The secondary ligand may be coupled with a suitable label and/or be the target (receptor) of tertiary ligand binding to the secondary ligand. The use of secondary, tertiary or even higher order ligands is often used to increase the signal. Suitable secondary and higher order ligands may include antibodies, secondary antibodies, and the well-known streptavidin-biotin system (Vector Laboratories, Inc.). The ligand or substrate may also be “tagged” with one or more tags as known in the art. Such tags may then be targets for higher order ligands. Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium, enzymatically active labels, radioactive labels, magnetic labels (“e.g. magnetic beads”, including paramagnetic and superparamagnetic labels), and fluorescent labels. Enzymatically active labels include e.g. horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star™ (Amersham Biosciences), ECF™ (Amersham Biosciences). A suitable enzyme-substrate combination may result in a colored reaction product, fluorescence or chemoluminescence, which can be measured according to methods known in the art (e.g. using a light-sensitive film or a suitable camera system). As for measuring the enyzmatic reaction, the criteria given above apply analogously. Typical fluorescent labels include fluorescent proteins (such as GFP and its derivatives), Cy3, Cy5, Texas Red, Fluorescein, and the Alexa dyes (e.g. Alexa 568). Further fluorescent labels are available e.g. from Molecular Probes (Oregon). Also the use of quantum dots as fluorescent labels is contemplated. Typical radioactive labels include 35S, 125I, 32P, 33P and the like. A radioactive label can be detected by any method known and appropriate, e.g. a light-sensitive film or a phosphor imager. Suitable measurement methods according the present invention also include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, or solid phase immune tests. Further methods known in the art (such as gel electrophoresis, 2D gel electrophoresis, SDS polyacrylamid gel electrophoresis (SDS-PAGE), Western Blotting, and mass spectrometry), can be used alone or in combination with labeling or other dectection methods as described above.

The amount of a peptide or polypeptide may be, also preferably, determined as follows: (a) contacting a solid support comprising a ligand for the peptide or polypeptide as specified above with a sample comprising the peptide or polypeptide and (b) measuring the amount peptide or polypeptide which is bound to the support. The ligand, preferably chosen from the group consisting of nucleic acids, peptides, polypeptides, antibodies and aptamers, is preferably present on a solid support in immobilized form. Materials for manufacturing solid supports are well known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The ligand or agent may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. Suitable methods for fixing/immobilizing the ligand are well known and include, but are not limited to ionic, hydrophobic, covalent interactions and the like. It is also contemplated to use “suspension arrays” as arrays according to the present invention (Nolan 2002, Trends Biotechnol. 20(1):9-12). In such suspension arrays, the carrier, e.g. a microbead or microsphere, is present in suspension. The array consists of different microbeads or microspheres, possibly labeled, carrying different ligands. Methods of producing such arrays, for example based on solid-phase chemistry and photo-labile protective groups, are generally known (U.S. Pat. No. 5,744,305).

The term “amount” as used herein encompasses the absolute amount of a polypeptide or peptide, the relative amount or concentration of the polypeptide or peptide as well as any value or parameter which correlates thereto or can be derived therefrom. Such values or parameters comprise intensity signal values from all specific physical or chemical properties obtained from the peptides by direct measurements, e.g., intensity values in mass spectra or NMR spectra. Moreover, encompassed are all values or parameters which are obtained by indirect measurements specified elsewhere in this description, e.g., response levels determined from biological read out systems in response to the peptides or intensity signals obtained from specifically bound ligands. It is to be understood that values correlating to the aforementioned amounts or parameters can also be obtained by all standard mathematical operations.

The term “comparing” as used herein encompasses comparing the amount of the peptide or polypeptide comprised by the sample to be analyzed with an amount of a suitable reference source specified elsewhere in this description. It is to be understood that comparing as used herein refers to a comparison of corresponding parameters or values, e.g., an absolute amount is compared to an absolute reference amount while a concentration is compared to a reference concentration or an intensity signal obtained from a test sample is compared to the same type of intensity signal of a reference sample. The comparison referred to in step (c) of the method of the present invention may be carried out manually or computer assisted. For a computer assisted comparison, the value of the determined amount may be compared to values corresponding to suitable references which are stored in a database by a computer program. The computer program may further evaluate the result of the comparison, i.e. automatically provide the desired assessment in a suitable output format. Based on the comparison of the amount determined in step a) and the reference amount, the risk of a cardiovascular event or mortality in a subject who suffers from the metabolic syndrome is predicted. Therefore, the reference amount is to be chosen so that either a difference or a similarity in the compared amounts allows allocation of subjects in groups with elevated, reduced or average risk.

Accordingly, the term “reference amount” as used herein refers to an amount which allows predicting the risk of mortality and/or a cardiovascular event for a subject suffering from the metabolic syndrome as referred to above. Accordingly, the reference may either be derived from (i) a sample of a subject known to have died or to have suffered from a cardiovascular event within a certain window period after the sample was obtained or (ii) a sample of a subject known not to have died and/or not to have suffered from a cardiovascular event within a certain window period after the sample was obtained. Moreover, the reference amount according to the invention may define a threshold amount, whereby an amount of both adiponectin and a natriuretic peptide larger than the threshold shall be indicative for a subject having an elevated risk of mortality and/or a cardiovascular event while an amount of adiponectin larger than the threshold and a natriuretic peptide lower than the threshold amount shall be an indicator for a subject which has a reduced risk of mortality and/or a cardiovascular event. Preferably, an amount of adiponectin lower than the threshold amount, regardless of the amount of a natriuretic peptide (thus the amount of a natriuretic peptide can be lower or larger than the threshold amount for the natriuretic peptide), shall be an indicator for a subject who is at average risk of mortality or suffering from a cardiovascular event.

The reference amount applicable for an individual subject may vary depending on various physiological parameters such as age, gender, or subpopulation, as well as on the means used for the determination of the polypeptide or peptide referred to herein. A suitable reference amount may be determined by the method of the present invention from a reference sample to be analyzed together, i.e. simultaneously or subsequently, with the test sample. A preferred reference amount serving as a threshold may be derived from the upper limit of normal (ULN), i.e. the upper limit of the physiological amount to be found in a population. The ULN for a given population of subjects can be determined by various well known techniques. A suitable technique may be to determine the median of the population for the peptide or polypeptide amounts to be determined in the method of the present invention.

Reference amounts of a diagnostic marker (i.e. of NTproBNP and adiponectin) can be established, and the level of the marker in a patient sample can simply be compared to the reference amount. The sensitivity and specificity of a diagnostic and/or prognostic test depends on more than just the analytical “quality” of the test-they also depend on the definition of what constitutes an abnormal result. In practice, Receiver Operating Characteristic curves, or “ROC” curves, are typically calculated by plotting the value of a variable versus its relative frequency in “normal” and “disease” populations. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results do not necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art. See, e.g., Hanley et al, Radiology 143: 29-36 (1982).

In certain embodiments, markers are selected to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement.

A reference amount defining a threshold amount for adiponectin, in particular high molecular weight adiponectin (in particular in a serum sample) as referred to in accordance with the present invention is, preferably 2.0 μg/ml, more preferably 2.4 μg/ml, or 2.8 μg/ml. The most preferred reference amount for adiponectin in the context of the present invention is 2.4 μg/ml (in particular in a serum sample).

A reference amount defining a threshold amount for a natriuretic peptide, in particular NT-proBNP, as referred to in accordance with the present invention is, preferably 30 pg/ml, more preferably 40 pg/ml or 50 pg/ml (thus, much lower than amounts of NT-proBNP that considered as reference amounts indicating a cardiovascular complication). The most preferred reference amount for NT-proBNP in the context of the present invention is 40 pg/ml (in particular in a serum sample).

Preferably, an amount of adiponectin and of NT-proBNP larger than the reference amounts is indicative for an elevated risk of mortality and/or a cardiovascular event. Preferably, an amount of adiponectin larger and an amount of NT-proBNP lower than the reference amounts is indicative for reduced risk of mortality and/or a cardiovascular event. More preferably, (i) an amount of adiponectin lower and (ii) an amount of NT-proBNP larger or lower than the reference amounts is indicative for average risk of mortality and/or a cardiovascular event.

Advantageously, it has been found in the studies underlying the present invention (see e.g. Examples) that determining the amount of adiponectin and a natriuretic peptide is required to reliably predict risk of mortality and/or a cardiovascular event in a subject suffering from the metabolic syndrome. Specifically, the amounts of NTproBNP and HMW adiponectin were determined in serum samples of healthy individuals and subjects suffering from the metabolic syndrome. It was shown that subjects suffering from the metabolic syndrome with increased adiponectin levels (larger than a reference amount, e.g. the median) had the largest long term risk of mortality and/or cardiovascular events if also the NT-proBNP level was increased (larger than the reference amount, e.g. the median), but had the lowest risk of mortality if the NT-proBNP level was decreased (lower than the median). Thus, depending on the NT-proBNP level, subjects with increased adiponectin levels could be allocated into two groups with opposed risks of mortality and/or cardiovascular events: into a low risk group and into a high risk group. Moreover, metabolic syndrome patients with decreased amounts of adiponectin were, regardless of their NT-proBNP levels, at average risk of mortality and/or of a cardiovascular event within a follow-up period of eight years. The results observed in the study underlying the present invention are surprising. In the art, the metabolic syndrome is associated with low adiponectin levels and it is generally thought that metabolic syndrome patients with low adiponectin levels have the worst outcome. Here, it was shown, that those subjects suffering from the metabolic syndrome are particularly at risk of mortality and/or of a cardiovascular event if the amount of adiponectin is increased, not decreased. It was shown that subject with increased amounts of adiponectin are at elevated risk if also the NT-proBNP level is increased. If the NT-proBNP level is decreased, those subjects have the best prognosis compared with other subjects suffering from the metabolic syndrome. These results are surprising since reduced levels of adiponectin in subjects with a metabolic syndrome were considered to be an indicator for an increased risk, not a decreased risk (see e.g. Trujillo M. E. et al.: “Adiponectin-journey from an adipocyte secretory protein to biomarker of the metabolic syndrome.” Journal of Internal Medicine Feb. 2005, vol. 257, no. 2, pp. 167-175). The terms increased marker level/amount, decreased marker level/amount, marker level/amount larger than, and marker level/amount smaller than, are meant to refer to an amount/level of marker molecule relative to the respective reference value.

Interestingly, in subject with decreased amounts of adiponectin, NT-proBNP does not play a significant role. Subjects suffering from the metabolic syndrome and with an adiponectin amount lower than a reference amount (here: the median), are at average risk of long term mortality and/or cardiovascular events, regardless of the amount of NT-proBNP. This is a very surprising result, since, in the art, it was thought that subjects with increased NT-proBNP levels are generally at significantly increased risk of dying and cardiovascular events than subjects with reduced NT-proBNP levels. This was, e.g., also observed for the control group. In the control group (subjects without a metabolic syndrome) analyzed in the context of the present invention, subjects with larger NT-proBNP levels were at significantly higher risk than subjects with lower NT-proBNP levels.

The method of the present invention will be, if applied, very beneficial since the method allows predicting the risk of mortality and/or cardiovascular events in subjects suffering from the metabolic syndrome. Depending on the risk, a suitable treatment can be initiated. The present invention is, particularly, advantageous for the identification of subjects which are susceptible to a therapy that aims to increase the adiponectin level. The data obtained in the context of the present invention strongly suggest that not all metabolic syndrome patients will benefit from a therapy which aims to increase the adiponectin level. Thus, the present invention therefore provides for an effective means which allows the physician to detect a risk of mortality and/or a cardiovascular event early so that the diagnosed patient may be subjected to an appropriate therapy which avoids or reduces the likelihood that the anticipated risk manifests itself. As a result, not only the individual patient profits from the present invention but society as a whole may safe costs for costly therapies.

Accordingly, the present invention relates to a method for identifying a subject being susceptible to a therapy for increasing the adiponectin level in a subject suffering from the metabolic syndrome, comprising the steps of:
a) determining, in a sample of the subject, the amount of adiponectin,
b) determining, in a sample of the subject, the amount of natriuretic peptide,
c) comparing the amounts as determined in step a) and b) to reference amounts, and
d) identifying a subject being susceptible to a therapy being capable of increasing the adiponectin level.

It is to be understood that the definitions and explanations of the terms made above and below apply mutatis mutandis for all embodiments/methods described in this specification and the accompanying claims except if the contrary is indicated.

The term “identifying” as used herein means assessing whether a subject who suffers from the metabolic syndrome will be eligible (and, thus susceptible) to a therapy for increasing the adiponectin level in a subject. It is to be understood that a subject who is susceptible to therapy, preferably, will benefit from the therapy. Particularly, the subject will have a reduced risk of mortality and/or a cardiovascular event as a consequence of the therapy. Moreover, a subject who is not susceptible to the therapy, preferably, will not benefit from the therapy. Particularly, the subject would have an increased risk of mortality and/or of a cardiovascular event as a consequence (adverse side effect) of therapy, or would have an unchanged risk of mortality or of a cardiovascular event. In the first case (increased risk), a therapy that could be harmful to the subject can be avoided when carrying out the method of the present invention, in the second case (unchanged), a therapy which would neither be particularly beneficial nor particularly harmful to the subject could be avoided, thereby having a positive impact on overall health care costs.

“A therapy for increasing the adiponectin level” as used herein, preferably, refers to any therapy that increases the adiponectin level, preferably the level of high molecular weight adiponectin in a subject. Such therapies for increasing the adiponectin level are well known in the art. Moreover, whether a certain therapy increases the level of adiponectin in a subject or no can be determined by the skilled person without any further ado. For example, the amount of adiponectin can be determined in a first sample of the subject, then a treatment to be tested for its effect on in adiponectin level can be initiated, then after the treatment has started (e.g. after one or two month) a second sample can be obtained, and the amount of adiponectin can be determined in the second sample. An increase, preferably a significant increase of the amount of the adiponectin in the second sample compared with the first sample, preferably, indicates that a certain therapy increases the adiponectin level in a subject. Preferably, a therapy which is capable of increasing the adiponectin level in a subject, is a therapy that increases the adiponectin level (preferably, the serum high molecular weight adiponectin level) significantly, more preferably by at least 10% to 15%,15 to 20%, 20 to 25%, 25 to 30%, 30% to 35%, 35% to 40% or more. Preferably, the increase is achieved during a time period of 3 months, 6 months, 9 months or 12 months. Most preferably, therapy increases the adiponectin level in s subject by 25 to 35% within a time period of 6 months. The publications of Kadowaki et al. (Journal of Clinical Investigation (2006), Adiponectin and adiponectin receptors in insulin resistance, diabetes, and the metabolic syndrome, vol. 117 (7):1784-1792) and Han et al. (Journal of the American College of Cardiology (2007), Adiponectin and Cardiovascular disease: response to therapeutic interventions, Vol. 49(5) 531-8) summarize therapies which aim to influence, particularly increase, the adiponectin level in a subject. Moreover, agents for increasing the adonectin level in a subject are disclosed in WO2007007732. The aforementioned documents are hereby incorporated in their entirety with respect to their disclosure content.

In the context of the present invention, therapy being capable of increasing the adiponectin level in a subject is, preferably, selected from the group consisting of significant weight reduction, preferably, of at least 10% of the body weight, more preferably of at least 15%, preferably by reduction of visceral adiposity, administration of ACE-inhibitors (particularly benazepril, captopril, cilazapril, enalapril, fosinopril, lisinopril, moexipril, perindopril, quinapril, ramipril, spirapril, and trandolapril), administration of peroxisome proliferator-activated receptor-alpha (PPAR alpha) agonists, preferably fibrates (particularly bezafibrate, ciprofibrate, clofibrate, gemfibrozil, fenofibrate), peroxisome proliferator-activated receptor-gamma (PPAR gamma) agonists (preferably thiazolidinediones such aus rosiglitazone and pioglitazone), administration of angiotensin II type I receptor blockers, administration of hypoglycaemic drugs (preferably glimepiride (but not metformin)) increasing linoleic acid intake Also contemplated as a therapy which is capable of increasing the adiponectin level in a subject is the administration of adiponectin. Particulary contemplated in the context of the present invention as a therapy being capable of increasing the adiponectin level in a subject is weight lost of at least 10% and the administration of thiazolidinediones, preferably rosiglitazone and pioglitazone. Thiazolidinediones are known to significantly upregulate adiponectin expression in white adipose tissue (see e.g. Maeda et al., Diabetes 2001. 50:2094-2099). Other pharmaceuticals for a therapy for increasing the adiponectin level are noacin and cannabinoid receptor antagonists such as rimonabant.

As will be understood by those skilled in the art, the aforementioned assessment is usually not intended to be correct for all (i.e. 100%) of the subjects to be identified. The term, however, requires that a statistically significant portion of subjects can be identified (e.g. a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. More preferably, at least 60%, at least 70%, at least 80% or at least 90% of the subjects of a population can be properly identified by the method of the present invention.

Preferably, the term “reference amounts” in the context of the aforementioned methods refers to amounts of the polypeptides which allows for identifying a subject (who suffers from the metabolic syndrome) being susceptible or not being susceptible to a therapy for increasing the amount of adiponectin. Accordingly, the reference may either be derived from (i) a subject known to be susceptible to therapy, particularly a subject known to have been successfully treated (particularly a subject who did not die or suffer from a cardiovascular event when getting the therapy) or (ii) a subject known not to be susceptible to therapy, particularly a subject known not to have been successfully treated (particularly a subject who died or suffer from a cardiovascular event as a consequence of the therapy). Preferred reference amounts can be found elsewhere herein.

Preferably, an amount of adiponectin, in a sample of a subject, lower than the reference amount for adiponectin and an amount of a natriuretic peptide lower than the reference amount for the natriuretic peptide indicates that the subject is susceptible to a therapy being capable of increasing the adiponectin level.

Preferably, an amount of adiponectin lower than the reference amount for adiponectin and an amount of a natriuretic peptide larger than the reference amount for the natriuretic peptide indicates that the subject is susceptible to a therapy being capable of increasing the adiponectin level. The subject is also susceptible to a cardiac therapy (for an explanation of this term, see below). In addition, the subject is, preferably, susceptible to a cardiac therapy (for an explanation of this term, see below).

Preferably, an amount of adiponectin larger than the reference amount for adiponectin, and an amount of a natriuretic peptide lower than the reference amount for the natriuretic peptide, indicates that the subject is not susceptible to a therapy being capable of increasing the adiponectin level. The subject, however, is, preferably, susceptible to a cardiac therapy (for an explanation of this term, see below).

Preferably, an amount of adiponectin larger than the reference amount for adiponectin, and an amount of a natriuretic peptide larger than the reference amount for the natriuretic peptide, indicates that the subject is not susceptible to a therapy being capable of increasing the adiponectin level.

The term “cardiac therapy” encompasses, preferably, those treatment regimens aim to ameliorate cardiac dysfunctions, preferably, heart failure. Preferably, the therapy envisages the administration of pharmaceuticals suitable for the treatment cardiac dysfunctions and, particularly, for heart failure. Pharmaceuticals suitable for the treatment of heart failure are well known in the art, see e.g. Heart Disease, 2005, 7th Edition, Eds. Braunwald, Elsevier Sounders, see tables 23-1, 23-6, 23-7, 23-8, 23-9, 23-10. Preferably, the administration of such pharmaceuticals aims to treat the symptoms and signs of heart failure and which aim to prevent a further progression of heart failure.

Drugs suitable for the treatment of heart failure are preferably, calcium antagonists (calcium channel blockers), digoxin, digitoxin, aldosterone antagonists, and diuretics.

Thus, the aforementioned method also allows the identification of subject which is susceptible to a cardiac therapy and, thus, will benefit from a cardiac therapy. Preferably, an amount of a natriuretic peptide larger than the reference amount indicates that the subject is susceptible, and, thus will benefit from a cardiac therapy. Preferably, if an amount of a natriuretic peptide lower than the reference amounts indicates that the subject is not susceptible to a cardiac therapy, and, thus, will not benefit the therapy (particularly as a consequence of increased health care costs and an increased risk of adverse site effects.

Thus, the present invention also relates to a method for assessing whether a subject suffering from the metabolic syndrome will benefit from a therapy for increasing the adiponectin level and/or a cardiac therapy, comprising the steps of:
a) determining, in a sample of the subject, the amount of adiponectin,
b) determining, in a sample of the subject, the amount of natriuretic peptide,
c) comparing the amounts as determined in step a) and b) to reference amounts, and
d) assessing whether the subject will benefit from therapy being capable increasing the adiponectin level and/or a cardiac therapy.

Preferably, a) an amount of adiponectin lower than the reference amount for adiponectin and an amount of a natriuretic peptide lower than the reference amount for the natriuretic peptide indicates that the subject will benefit from a therapy being capable of increasing the adiponectin level, and that the subject will not benefit from a cardiac therapy; and

b) an amount of adiponectin lower than the reference amount for adiponectin and an amount of a natriuretic peptide larger than the reference amount for the natriuretic peptide indicates that the subject will benefit from a therapy being capable of increasing the adiponectin level, and will also benefit from a cardiac therapy; and

c) an amount of adiponectin larger than the reference amount for adiponectin, and an amount of a natriuretic peptide larger than the reference amount for the natriuretic peptide, indicates that the subject will not benefit from a therapy being capable of increasing the adiponectin level, and will benefit from a cardiac therapy; and

d) an amount of adiponectin larger than the reference amount for adiponectin, and an amount of a natriuretic peptide lower than the reference amount for the natriuretic peptide, indicates that the subject will not benefit from a therapy being capable of increasing the adiponectin level and that the subject will not benefit from a cardiac therapy.

Preferred reference amounts are described elsewhere herein. Surprisingly, the results obtained in the context of the present invention indicate that subjects with a metabolic syndrome with slightly increased amounts of a natriuretic peptide (particularly NT-proBNP larger than 40 μg/ml) are at increased risk and therefore would benefit from a cardiac therapy. In the art, the reference amount for NT-proBNP indicating the need of a cardiac therapy is generally much larger (e.g. 125 μg/ml) than the reference amount for NT-proBNP according to the present invention.

Moreover, the present invention also envisages kits and devices adapted to carry out the method of the present invention.

Accordingly, the present invention relates to a device for predicting the risk of mortality and/or a cardiovascular event in a subject suffering from metabolic syndrome, comprising means for determining the amount of adiponectin and a natriuretic peptide in a sample of a subject, and means for comparing the amount of adiponectin and a natriuretic peptide determined by the means with reference amounts, allowing predicting the risk of mortality and/or a cardiovascular event in a subject suffering from metabolic syndrome.

Also envisaged by the present invention is a device for identifying a subject being susceptible to a therapy being capable of increasing the adiponectin level in a subject, comprising means for determining the amount of adiponectin and a natriuretic peptide in a sample of a subject suffering from the metabolic syndrome, and means for comparing the amount of adiponectin and a natriuretic peptide determined by the means with reference amounts, allowing identifying a subject being susceptible to a therapy being capable of increasing adiponectin level in the subject.

The term “device” as used herein relates to a system of means comprising at least the aforementioned means operatively linked to each other as to allow the identification of subjects being susceptible to a therapy for increasing the adiponectin level in the subject or for predicting the risk of mortality and/or an cardiovascular event for a subject suffering from the metabolic syndrome. Preferred means for determining the amount of a adiponectin and a natriuretic peptide, and means for carrying out the comparison are disclosed above in connection with the method of the invention. How to link the means in an operating manner will depend on the type of means included into the device. For example, where means for automatically determining the amount of the peptides are applied, the data obtained by the automatically operating means can be processed by, e.g., a computer program in order to obtain the desired results. Preferably, the means are comprised by a single device in such a case. The device may accordingly include an analyzing unit for the measurement of the amount of the peptides or polypeptides in an applied sample and a computer unit for processing the resulting data for the evaluation. Alternatively, where means such as test strips are used for determining the amount of the peptides or polypeptides, the means for comparison may comprise control strips or tables allocating the determined amount to a reference amount. The test strips are, preferably, coupled to a ligand which specifically binds to the peptides or polypeptides referred to herein. The strip or device, preferably, comprises means for detection of the binding of the peptides or polypeptides to the ligand. Preferred means for detection are disclosed in connection with embodiments relating to the method of the invention above. In such a case, the means are operatively linked in that the user of the system brings together the result of the determination of the amount and the diagnostic or prognostic value thereof due to the instructions and interpretations given in a manual. The means may appear as separate devices in such an embodiment and are, preferably, packaged together as a kit. The person skilled in the art will realize how to link the means without further ado. Preferred devices are those which can be applied without the particular knowledge of a specialized clinician, e.g., test strips or electronic devices which merely require loading with a sample. The results may be given as output of raw data which need interpretation by the clinician. Preferably, the output of the device is, however, processed, i.e. evaluated, raw data the interpretation of which does not require a clinician. Further preferred devices comprise the analyzing units/devices (e.g., biosensors, arrays, solid supports coupled to ligands specifically recognizing the polypeptide whose amount shall be determined, Plasmon surface resonance devices, NMR spectrometers, mass-spectrometers etc.) or evaluation units/devices referred to above in accordance with the method of the invention.

Moreover the present invention is concerned with a kit adapted to carry out the method of the present invention, and thus for predicting the risk of mortality and/or a cardiovascular event in a subject suffering from metabolic syndrome, the kit comprising instructions for carrying out the method, and means for determining the amount of adiponectin and a natriuretic peptide in a sample of a subject suffering from the metabolic syndrome, and means for comparing the amount of adiponectin and a natriuretic peptide determined by the means with reference amounts, allowing predicting the risk of mortality and/or a cardiovascular event in a subject suffering from metabolic syndrome.

Moreover, the present invention relates to kit adapted to carry out the method of the present invention, and, thus, for identifying a subject being susceptible to a therapy being capable of increasing the adiponectin level in a subject, the kit comprising instructions for carrying out the method, and means for determining the amount of adiponectin and a natriuretic peptide in a sample of a subject suffering from the metabolic syndrome, and means for comparing the amount of adiponectin and a natriuretic peptide determined by the means with reference amounts, allowing assessing whether a subject is susceptible to a therapy capable of increasing the adiponectin level.

The term “kit” as used herein refers to a collection of the aforementioned compounds, means or reagents of the present invention which may or may not be packaged together. The components of the kit may be comprised by separate vials (i.e. as a kit of separate parts) or provided in a single vial. Moreover, it is to be understood that the kit of the present invention is to be used for practicing the methods referred to herein above. It is, preferably, envisaged that all components are provided in a ready-to-use manner for practicing the methods referred to above. Further, the kit preferably contains instructions for carrying out the methods. The instructions can be provided by a users manual in paper- or electronic form. For example, the manual may comprise instructions for interpreting the results obtained when carrying out the aforementioned methods using the kit of the present invention.

All references cited in this specification are herewith incorporated by reference with respect to their entire disclosure content and the disclosure content specifically mentioned in this specification.

The following Examples shall merely illustrate the invention. They shall not be construed, whatsoever, to limit the scope of the invention.

### EXAMPLE 1

### Determination of High Molecular Weight Adiponectin and NT-proBNP in Samples of Patients Suffering from the Metabolic Syndrome

High molecular weight adiponectin and NT-proBNP were determined in serum samples obtained from a total of 2656 randomly selected subjects as well as in serum samples of 356 subjects with the metabolic syndrome. The 356 subjects suffering from the metabolic syndrome were selected according to criteria for the presence of the metabolic syndrome as defined by the European Group for the study of insulin resistance (EGIR, serum insulin larger 44 pmol/1, together with two of the following criteria: BMI larger than 30 kg/m2, serum triglycerides larger 2 mmol/1 or S-HDL lower 1 mmol/1, Glucose larger 6.1 mmol/l, blood pressure larger 140 systolic/90 diastolic mm Hg). HMW adiponectin was determined with the Adiponectin (Multimeric) EIA kit (Alpco Diagnostics, Salem, USA Catalog Number: 47-ADPH-9755). Medians for NT-proBNP and adiponectin and for the both groups (healthy individuals, individuals with metabolic syndrome were determined):

| | |
|---|---|
| Healthy individuals: | NTproBNP 53 pg/ml, |
| | adiponectin 3.6 μg/ml; |
| Individuals with metabolic syndrome: | NTproBNP 39.7 pg/ml, |
| | adiponectin 2.38 μg/ml. |

In a follow up study, the mortality in eight years after obtaining the samples was determined (see Table 1).

TABLE 1
Mortality during a follow up period of eight years

| | | Mortality: | | |
|---|---|---|---|---|
| | | Metabolic | | Mortality: |
| Adiponectin | NT-proBNP | syndrome | | Healthy |
| level | level | group (risk) | | subjects |
| Larger than | Lower than | 9.5% | (low) | 5.6% |
| median | median | | | |
| Larger than | Larger than | 22% | (high) | 13.2% |
| median | median | | | |
| Lower than | Lower than | 15.1% | (average) | 6.2% |
| median | median | | | |
| Lower than | Larger than | 15.4% | (average) | 11% |
| median | median | | | |

### EXAMPLE 2

### Individual Case Studies

A 52 year-old female obese patient (BMI: 34, blood pressure 145/90 mm Hg, metabolic syndrome) who otherwise does not have any discomforts presents at her family doctor. A routine examination is carried out (chest x-ray, ECG, stress ECG) indicating the presence of a left ventricular hypertrophy. The serum levels of HMW adiponectin (3.8 μg/ml) and NT-proBNP (215 μg/ml) are significantly increased. The patient is advised to lose weight. ACE inhibitors, a beta blocker and a mild diuretic are prescribed. However, after 15 months the patient develops an ACE (Non-STEMI with a troponin T level of 0.5 μg/l). An angiography is carried out showing a significant arteriosclerosis. The case shows the predictive value of the method of the present invention. The patient has a metabolic syndrome and has serum adiponectin and NT-proBNP levels larger than the reference amount (median) and, therefore, an increased risk of mortality and/or a cardiovascular event. 15 months after the initial examination, the patient suffers from a cardiacvascular event. This is surprising since several studies have shown that increased adiponectins level are a associated with a lower risk of cardiovascular events (see, e.g. Kumada et al., Pischon et al. and Inoue et al., loc. cit.). In the studies underlying the present invention, however, it has been shown, that increased adiponectin levels in patients with a metabolic syndrome are associated with an elevated risk of cardiovascular events, if the patient also has increased NT-proBNP.

A 48 year-old male patient who has overweight (body mass index 32), but otherwise feels well shows up at his family doctor. A routine examination is carried out including an ECG and an x-ray of the thorax (without any pathological findings). The blood pressure is 150/95. However, a metabolic syndrome is diagnosed. HMW Adiponectin (1.8 μg/ml and NT-proBNP (43 pg/ml) are determined in a serum sample obtained from the patient. In the following years, there are no significant changes regarding the serum levels of HMW Adiponectin and NT-proBNP. However, the patient is advised to lose weight. Due to the results of a glucose stress test, the patient further is advised to take rosiglitazone. After 4.5 years, he shows symptoms of ACS (but has no myocardial infarction). A coronary angiography is carried out indicating 50% stenosis of the right coronary artery. The case shows the predictive value of the method of the present invention. The patient has a serum adiponectin level lower than the reference amount (median) and a serum NT-proBNP levels larger than the reference amount (median). Therefore, the patient is at average risk of mortality and/or a cardiovascular event. 2.5 years after the initial examination, the patient suffers from a cardiovascular event.

A 53 year-old male patient (with a metabolic syndrome) shows up at his family doctor for a routine examination. ECG, chest x-ray of the thorax and stress ECG are normal (i.e. no pathological findings). HMW Adiponectin (4.1 μg/ml) and NT-proBNP (26 pg/ml) are determined in a serum sample obtained from the patient. Within the following five years, there are no changes regarding these tests. Moreover, there is no acute cardiac event within the following five years. This case shows that a patient with a metabolic syndrome with an adiponectin level larger than the reference amount (median) and a NT-proBNP level lower than the reference amount (median) is at reduced risk of mortality and/or of a cardiovascular event.

### EXAMPLE 3

### Patient without a Metabolic Syndrome

A 46 year-old male patient (no overweight, blood pressure 125/75 mm Hg, no metabolic syndrome) presents at his family doctor for a routine check-up. An ECG and an x-ray of the thorax, a stress ECG, and a echocardiogram are carried out however, without any pathological findings. HMW Adiponectin (3.1 μg/ml) and NT-proBNP (47 pg/ml) are determined in a serum sample obtained from the patient. Within the following 5 years, there are no significant changes regarding these tests. Moreover, there are no chances regarding the ECG, the stress ECG and the chest x-ray. No ACS is observed within the following five years.

What is claimed is:

1. A method for predicting a risk of mortality and/or a cardiovascular event in a subject suffering from metabolic syndrome comprising the steps of:
determining an amount of adiponectin in a sample from the subject,
determining an amount of a natriuretic peptide in a sample from the subject,
comparing the amount of adiponectin and the natriuretic peptide determined to reference amounts for adiponectin and the natriuretic peptide, and
predicting the risk of mortality and/or cardiovascular event in the subject wherein
an amount of the natriuretic peptide larger than the reference amount for the natriuretic peptide, and an amount of adiponectin larger than the reference amount for the adiponectin indicates that the subject is at elevated risk of mortality and/or cardiovascular event,
an amount of the natriuretic peptide lower than the reference amount for the natriuretic peptide, and an amount of adiponectin larger than the reference amount for the adiponectin indicates that the subject is at reduced risk of mortality and/or cardiovascular event, and
an amount of the natriuretic peptide lower or larger than the reference amount for the natriuretic peptide, and an amount of adiponectin lower than the reference amount of the adiponectin indicates that the subject is at average risk of mortality and/or cardiovascular event.

2. The method of claim 1, wherein the sample is selected from the group consisting of blood, blood serum, and blood plasma.

3. The method of claim 1, wherein the adiponectin is high molecular weight adiponectin.

4. The method of claim 1, wherein the natriuretic peptide is N-terminal pro-brain natriuretic peptide (NT-pro BNP).

5. The method of claim 1, wherein the sample is serum, the adiponectin is high molecular weight adiponectin, and the reference amount for adiponectin is 2.4 μg/ml.

6. The method of claim 1, wherein the sample is serum, the adiponectin is high molecular weight adiponectin, and the reference amount for adiponectin is 2.4±0.2 μg/ml.

7. The method of claim 1, wherein the sample is serum, the adiponectin is high molecular weight adiponectin, and the reference amount for adiponectin is 2.4±0.5 μg/ml.

8. The method of claim 1, wherein the sample is serum, the natriuretic peptide is N-terminal pro-brain natriuretic peptide (NT-pro BNP), and the reference amount for NT-proBNP is 40 pg/ml.

9. The method of claim 1, wherein the sample is serum, the natriuretic peptide is N-terminal pro-brain natriuretic peptide (NT-pro BNP), and the reference amount for NT-proBNP is 40±3 pg/ml.

10. The method of claim 1, wherein the sample is serum, the natriuretic peptide is N-terminal pro-brain natriuretic peptide (NT-pro BNP), and the reference amount for NT-proBNP is 40±5 pg/ml.

11. A method for assessing whether a subject suffering from metabolic syndrome will benefit from a therapy from an increasing adiponectin level, the method comprising the steps of:
determining an amount of adiponectin in a sample from the subject,
determining an amount of a natriuretic peptide in a sample from the subject,
comparing the amount of adiponectin and the natriuretic peptide determined to reference amounts for adiponectin and the natriuretic peptide, and
assessing whether the subject will benefit from the therapy wherein
an amount of adiponectin lower than the reference amount for adiponectin and an amount of the natriuretic peptide lower than the reference amount for the natriuretic peptide indicates that the subject is susceptible to a therapy capable of increasing the adiponectin an amount of adiponectin lower than the reference amount for adiponectin and an amount of the natriuretic peptide larger than the reference amount for the natriuretic peptide indicates that the subject is susceptible to a therapy capable of increasing the adiponectin level,
an amount of adiponectin larger than the reference amount for adiponectin, and an amount of the natriuretic peptide lower than the reference amount for the natriuretic peptide, indicates that the subject is not susceptible to a therapy capable of increasing the adiponectin level, or
an amount of adiponectin larger than the reference amount for adiponectin, and an amount of the natriuretic peptide larger than the reference amount for the natriuretic peptide, indicates that the subject is not susceptible to a therapy capable of increasing the adiponectin level.

12. The method of claim 11, wherein therapy capable of increasing the adiponectin level in the subject is selected from the group consisting of weight reduction of at least 10%, preferably by reduction of visceral adiposity, administration of angiotensin-converting enzyme (ACE) inhibitors, administration of peroxisome proliferator-activated receptor-alpha (PPAR alpha) agonists, peroxisome proliferator-activated receptor-gamma (PPAR gamma) agonists, administration of hypoglycaemic drugs, and increasing linoleic acid intake.

13. The method of claim 12, wherein the therapy is administration of thiazolidinediones.

14. A device for predicting a risk of mortality and/or a cardiovascular event in a subject suffering from metabolic syndrome, the device comprising
a means for determining an amount of adiponectin and a natriuretic peptide in a sample from the subject, and
a means for comparing the amounts of adiponectin and the natriuretic peptide determined with reference amounts for the adiponectin and the natriuretic peptide, allowing prediction of the risk of mortality and/or cardiovascular event in the subject.

15. A kit adapted for predicting a risk of mortality and/or a cardiovascular event in a subject suffering from metabolic syndrome according to the method of claim 1, the kit comprising instructions for carrying out the method,
a means for determining an amount of adiponectin and an amount of a natriuretic peptide in a sample from the subject, and
a means for comparing the amounts of adiponectin and the natriuretic peptide determined with reference amounts for the adiponectin and the natriuretic peptide, allowing prediction of the risk of mortality and/or a cardiovascular event in a subject.

16. A device for assessing whether a subject suffering from metabolic syndrome will benefit from a therapy for increasing an adiponectin level in the subject, the device comprising
a means for determining an amount of adiponectin and a natriuretic peptide in a sample from the subject, and
a means for comparing the amounts of the adiponectin and the natriuretic peptide determined with reference amounts for the adiponectin and the natriuretic peptide, allowing assessing whether a subject will benefit from a therapy for increasing the adiponectin level.

17. A kit adapted for assessing whether a subject suffering from metabolic syndrome will benefit from a therapy from an increasing adiponectin level according to the method of claim 11, the kit comprising
instructions for carrying out the method,
a means for determining an amount of adiponectin and a natriuretic peptide in a sample from the subject, and
means for comparing the amounts of adiponectin and natriuretic peptide determined with reference amounts for the adiponectin and natriuretic peptide, thereby allowing assessment whether a subject will benefit from a therapy for increasing the adiponectin level.
